(19) United States
(12) Patent Application Publication
(10) Pub. No.: US 2025/0281529 A1
(43) Pub. Date: Sep. 11, 2025

(54) TREATMENT OF DISEASES AND CONDITIONS OF THE EYE

(71) Applicant: Tufts Medical Center, Inc., Boston, MA (US)

(72) Inventor: Pedram HAMRAH, Wellesley, MA (US)

(21) Appl. No.: 18/861,256

(22) PCT Filed: May 1, 2023
(86) PCT No.: PCT/US2023/020621

§ 371 Date: Oct. 29, 2024

Related U.S. Application Data
(60) Provisional application No. 63/336,902, filed on Apr. 29, 2022.

(51) Int. Cl. A61K 35/15; A61K 9/00; A61K 45/06; A61P 27/02; A61P 37/06

(52) U.S. Cl. CPC A61K 35/15; A61K 9/0048; A61K 45/06; A61P 27/02; A61P 37/06

(57) ABSTRACT

The invention provides methods and compositions for use in the treatment and prevention of diseases and conditions of the eye.

### STATEMENT AS TO FEDERALLY FUNDED RESEARCH

This invention was made with government support under grant number EY029602 awarded by the National Institutes of Health. The government has certain rights in the invention.

### FIELD OF THE INVENTION

The invention relates to methods and compositions for use in the treatment and prevention of diseases and conditions of the eye.

### BACKGROUND

Diseases of the eye can be very uncomfortable and painful, leading to adverse impacts on quality of life. As one example, dry eye disease (DED) is a common condition in which tears produced in the eye are not able to provide adequate moisture to the eye, which may thus become dry, red, and inflamed. If left untreated, severe DED may lead to eye infections, bacterial keratitis, eye inflammation, abrasion of the corneal surface, corneal ulcers and erosion, scarring of the eye, and vision loss. Neuropathic corneal pain (NCP) is a condition characterized by unremitting eye sensitivity with ocular pain, light sensitivity, or burning. NCP shares some features with DED, and indeed is sometimes misdiagnosed as DED, but it does not respond to conventional treatment that may be effective in treating DED (e.g., artificial tears). The cause of NCP can vary from past herpetic infections, recurrent corneal erosions, certain medications, cataract, or refractive laser surgery, combined with inflammation, or from systemic diseases, such as small fiber neuropathy, diabetic neuropathy, anxiety, depression, or migraines. These causes all affect the corneal nerves, possibly leading to irreversible damage or dysfunction. Neurotrophic keratopathy (NK), or neurotropic keratitis, is a degenerative disease characterized by decreased corneal sensitivity and poor corneal healing. NK leaves the cornea susceptible to injury and decreases reflex tearing. NK can result in severe ocular surface disease, neurotrophic corneal ulcers, or corneal melting.

There is a need for improved treatments for diseases and conditions of the eye including, e.g., DED, NCP, and NK.

### SUMMARY

In a first aspect, the invention provides a method of treating a disease or condition of the eye in a subject, the method comprising administering a plasmacytoid dendritic cell (pDC) secretome to the eye of the subject.

In a second aspect, the invention provides a method of increasing nerve density in the eye of a subject, the method comprising administering a pDC secretome to the eye of the subject.

In a third aspect, the invention provides a method of improving corneal nerve function in a subject, the method comprising administering a pDC secretome to the eye of the subject.

In a fourth aspect, the invention provides a method of promoting corneal nerve survival in a subject, the method comprising administering a pDC secretome to the eye of the subject.

In a fifth aspect, the invention provides a method of treating a subject who has undergone corneal transplantation, the method comprising administering a pDC secretome to the eye of the subject post transplantation to prevent rejection.

The following embodiments each apply, independently, to the different aspects described herein.

In some embodiments, the method prevents, reduces, or eliminates one or more symptoms of a disease or condition of the eye in the subject.

In some embodiments, the subject has or is at risk of developing dry eye disease (DED), neuropathic corneal pain (NCP), chronic ocular service pain (COSP), neurotrophic keratopathy (NK), corneal neovascularization, persistent corneal epithelial defect, or one or more other diseases or conditions characterized by ocular nerve degeneration or damage, ocular inflammation, or ocular neovascularization (e.g., as described herein).

In some embodiments, the pDC secretome composition is administered to the eye of the subject by eye drops, ointment, gel, polymers, or injection.

In some embodiments, the method does not comprise debridement of the corneal surface of the eye.

In some embodiments, the pDC secretome is acellular.

In some embodiments, the pDC secretome comprises one or more of brain-derived neurotrophic factor (BDNF), glial cell derived neurotrophic factor (GDNF), neurotrophin-4/5 (NT-4/5), nerve growth factor (NGF), anti-inflammatory molecules, and anti-angiogenic molecules.

In some embodiments, the pDC secretome comprises each of BDNF, GDNF, NT-4/5, NGF, anti-inflammatory molecules, and anti-angiogenic molecules.

In some embodiments, the pDC secretome functions in the presence of anti-NGF, and optionally the method is carried out for the treatment of neuropathic pain.

In some embodiments, the method further comprises administration of an additional therapeutic agent to the eye of the subject.

In some embodiments, the additional therapeutic agent is a TLR7 agonist, a TLR9 agonis, or nerve growth factor (NGF).

In some embodiments, the subject is a human.

In some embodiments, the subject is a veterinary subject, such as a dog or a cat.

In some embodiments, the pDC secretome comprises molecules secreted from pDCs cultured in a cell culture medium, and said cell culture medium (cell culture supernatant) comprising said molecules is administered to said subject.

In a sixth aspect, the invention provides a pharmaceutical composition comprising a pDC secretome, which optionally is in dosage form for administration to a subject.

In some embodiments, the pDC secretome comprises molecules secreted from pDCs cultured in a cell culture medium, and said pDC secretome is present within said cell culture medium (cell culture supernatant).

In a seventh aspect, the invention provides a pharmaceutical kit comprising a pDC secretome in dosage form, and optionally a vessel and/or device for use in administration (e.g., an eye dropper).

In some embodiments, the pDC secretome comprises molecules secreted from pDCs cultured in a cell culture medium, and said pDC secretome is present within said cell culture medium (cell culture supernatant).

In an eighth aspect, the invention provides a method of generating a pDC secretome for use in treating a disease or condition of the eye, the method comprising culturing pDCs in a medium and removing the pDCs from the medium.

In some embodiments, the method further comprises collecting said medium for use as a composition comprising said pDC secretome in said treatment.

In some embodiments, the method further comprises freezing or lyophilizing the medium after pDC removal.

Also included in the invention are use of the compositions described herein for the treatment of the diseases and conditions described herein.

The invention provides several advantages. For example, the compositions used in the invention include multiple growth factors, and thus will thus be more effective than treatment with a single growth factor (e.g., nerve growth factor). Furthermore, in contrast to cell-based therapies, there is no requirement for a step of debridement of the corneal surface for administration of the acellular compositions of the present invention. This results in substantial benefits with respect to patient comfort and compliance. Also, in contrast to cell-based therapies, the methods of the invention do not require the use of autologous cells, which are obtained from the person to whom they are to be administered. Rather, the compositions of the invention can be obtained using any pDCs, isolated from essentially any person. This results in substantial benefits with respect to efficiency, cost, and compliance. In addition, compositions of the invention can be administered by a patient themselves at home or by medical paraprofessionals, without the need for complex clinical intervention. The invention further provides an approach that does not require injection or other clinical steps that may discourage a patient from seeking treatment. The invention additionally provides an approach that does not involve the administration of living cells to a subject, which some subjects may prefer. Furthermore, the approach of the invention causes increased expression of neurotrophic molecules in the eye, leading to increased nerve density, which in turn can lead to permanent improvements in ocular health.

Other features and advantages of the invention will be apparent from the following detailed description, the drawings, and the claims.

### BRIEF DESCRIPTION OF THE DRAWINGS

FIG. 1 is a series of confocal micrographs (A-E) and graphs (F-H) showing the effects of topical application of the pDC secretome on corneal nerve loss.

FIG. 2 shows a sample trace of ex vivo corneal electrophysiology.

FIG. 3 is a series of graphs showing electrophysiological recordings of corneal nerve terminal impulses, which reveal dysfunction of high-threshold cold receptors following pDC depletion.

FIG. 4 is a graph showing hyperosmolar saline response, as detected by paw wipes/30 seconds, in ciliary nerve-ligated mice treated with vehicle, or pDC supernatant.

FIG. 5 is a graph showing palpebral opening in ciliary nerve-ligated mice treated with pDC supernatant.

FIG. 6 is a set of plots showing that plasmacytoid dendritic cells (pDCs) express neurotrohic molecules.

FIG. 7 is a set of photographs showing the results of pDC supernatant treatment in a model of DED.

FIG. 8 is a series of graphs showing the effects of pDC supernatant treatment on tear secretion volume (A), corneal fluorescein score (B), Cochet-Bonnet esthesiometry (C), and number of eye wipes per 30 seconds (D).

FIG. 9 is a series of graphs showing the mRNA levels of NGF, BDNF, and NT3 in naïve, medium only treated, and pDC supernatant treated mice.

FIG. 10 is a graph showing corneal nerve hypersensitivity (hyperalgesia) responses to 5M saline following sham surgery and ciliary nerve ligation model of neuropathic corneal pain with or without treatments (pDC secretome or pDC transfer).

FIG. 11 is a series of graphs showing the effects of pDC supernatant and pDC adoptive transfer in a murine model of dry eye disease.

FIG. 12 is a series of photographs and a series of graphs showing that NGF is not a required component of the pDC secretome for obtaining beneficial effects.

### DETAILED DESCRIPTION

The invention provides methods and compositions for use in the treatment of diseases and conditions of the eye including, for example, dry eye disease (DED), neuropathic corneal pain (NCP), chronic ocular service pain (COSP), neurotrophic keratopathy (NK), corneal neovascularization, and persistent corneal epithelial defect (also see below). The methods and compositions of the invention can further be used to increase nerve density in the eye, improve corneal nerve function, and promote corneal nerve survival. Furthermore, the methods and compositions can be used to treat subjects who have undergone corneal transplantation, in order to prevent rejection.

The compositions of the invention comprise components of the secretome of plasmacytoid dendritic cells (pDCs). The compositions therefore comprise molecules that are secreted from cultured pDCs into the liquid medium in which they are cultured. The compositions are referred to herein as pDC secretome compositions, and also can be referred to as pDC supernatants, pDC culture medium, or pDC conditioned medium. In preferred embodiments, the compositions of the invention are acellular and thus do not include pDCs.

The present application shows, for the first time, that pDC secretome compositions administered to the eye can have clinically meaningful effects including, for example, improvements in several features of DED, NCP, COSP, NK, corneal neovascularization, and persistent corneal epithelial defect, as well as result in increases in ocular nerve density. The results described herein bring the field forward as they provide a basis for therapeutic approaches that are effective, safe, and comfortable for subjects. In addition, it was surprising that pDC supernatants include sufficient active agents such that administration can be effective, and yet not overly cumbersome in terms of optimal frequency of administration. Furthermore, secretome use allows for the treatment of diseases such as those described herein without the need for epithelial debridement and without direct transfer of cells to the eye. The secretome use further allows for treatment without the need for the use of tissue glue.

The compositions and methods of the invention are described further below.

### Compositions and Methods

The cells used to generate the compositions of the invention are plasmacytoid dendritic cells (pDCs), which circulate in the blood and can also be found in peripheral lymphoid organs. pDCs are bone marrow-derived innate immune cells that express Toll-like receptors (TLR) 7 and 9. In mice, they express low levels of CD11c, which differentiates them from conventional dendritic cells (cDCs), and exhibit PDCA-1, Siglec-H, and CD45R/B220. In humans, pDCs are positive for blood-derived dendritic cell antigen (BDCA)-2 (CD303), BDCA-4 (CD304), and CD123. Upon activation, they produce large amounts of type 1 interferons (see, e.g., Tversky et al., Clin. Exp. Allergy 38(5):781-788, 2008; Asselin-Paturel et al., Nat. Immunol. 2(12):1144-1150, 2001; Nakano et al., J. Exp. Med. 194(8):1171-1178, 2001; Bjorck, Blood 98(13):3520-3526, 2001).

pDCs for use in generating the compositions of the invention can be isolated from a subject to whom the compositions are to be administered or they can be obtained from a donor (e.g., a human donor). Accordingly, the source may be autologous or allogeneic. In some embodiments, the pDCs are obtained from blood, which may optionally be obtained from a blood bank. In other embodiments, the pDCs are obtained from bone marrow, a cell line generated from human pDCs, and/or humanized pDCs. Because the cells themselves are not administered, the source of the cells is not of central importance which, as explained above, is advantageous. pDCs can be isolated from blood or bone marrow using standard techniques including, e.g., density gradient centrifugation and marker-based cell separation. Optionally, the pDCs can be cultured and/or frozen prior to use in generating the compositions of the invention. Furthermore, the pDCs can be obtained by the stimulation of cultured bone marrow cells. For example, peripheral blood mononuclear cells (PBMCs) can be isolated from blood using, e.g., FICOLL® (neutral, highly branched, high-mass hydrophilic polysaccharide; GE Healthcare) gradient density centrifugation. Then, pDCs can be isolated from PBMCs based on a pDC-specific or pDC-enriched marker (e.g., BDCA-2, BDCA-4, or CD123). An antibody against such a marker (e.g., an anti-BDCA-2, anti-BDCA-4, or anti-CD123 antibody) can be used in this isolation step using standard methods (e.g., microbead or magnetic bead-based separation or fluorescence-activated cell sorting [FACS]).

In a specific, non-limiting example of obtaining pDCs, 5-10 ml blood is collected from a subject or a donor via routine venipuncture and is placed in a tube containing citrate as an anti-coagulant. Next, PBMCs are separated by standard Percoll density gradient centrifugation. After isolating PBMCs, pDCs are selected via commercially available magnetic beads according to the manufacturer's instructions (Miltenyi Biotec). In brief, PBMCs are blocked with an anti-Fc receptor antibody for 15 minutes at room temperature (RT). Next, samples are labeled by incubation with an anti-BDCA2 antibody conjugated with microbeads for 30 minutes at 4° C. Cells labeled with magnetic bead-conjugated BDCA-2 antibodies (which will constitute pDCs) are then applied to a separation column, placed in a separation device standing on a magnetic field. By washing the separation column with sterile washing buffer, BDCA2-negative cells (non-pDCs) are washed out, while BDCA-2⁺ labeled pDCs stay attached to the column. At this step, the separation column is removed from the magnetic field and pDCs are eluted by pushing washing buffer through the column. After separation, the number of pDCs can be determined by routine trypan blue staining on a portion of collected cells and the purity of the sample can be measured by immunofluorescence staining with a BDCA2 fluorochrome-conjugated antibody (as well as other human pDC markers including BDCA-4 and CD123, if needed) and analyzed with FACS. In case analysis shows that additional purity of the isolated cells may be desired (e.g., if less than 85% purity is found), then purity can be improved by another optional round of magnetic separation. Cells can then be centrifuged and resuspended in culture medium for the generation of compositions of the invention.

pDCs can be cultured in any of a number of different types of media that are known in the art for the culture of immune cells, such as dendritic cells (e.g., pDCs). In some embodiments, the medium is serum-free medium. In one specific, non-limiting example, the medium is serum-free RPMI 1640 medium (Gibco, Invitrogen, Corning), which includes L-glutamine, and is supplemented with penicillin (50 U/mL)/streptomycin (50 ug/mL). The components of Corning RMPI 1640 are provided further below in Table 1 for illustrative purposes. pDCs can be cultured for a predetermined amount of time and/or to reach a particular density or density range. For example, the cells can be cultured to a density ranging from 1×10⁵ to 1×10⁷ cells/mL, e.g., to about 1×10⁶ cells/mL. Culture is preferably carried out at 34° C.-40° C., e.g., 36° C.-38° C., 36° C.-37° C., or about 37° C. Culture typically is carried out in 4-10%, e.g., 5-7% CO₂, depending upon the buffering agent that may be present in the medium. Furthermore, the pH of the culture medium can be, for example, pH 7.0-8.0, e.g., pH 7.4-7.7. Once cells reach a desired density, e.g., 1×10⁵ to 1×10⁷ cells/mL, or about 1×10⁶ cells/mL, the cells are pelleted and the medium is decanted for use in the invention. The medium can optionally be frozen before thawing and maintenance at 4° C. before use.

In a specific, non-limiting example, pDCs are cultured in either tissue culture tubes (Falcon) or 24-well culture plates (Falcon) at a density of about 1×10⁶ cells/mL in a maximum volume of 1 mL per tube or well. Cultures are maintained in a 37° C. incubator, with 5% CO₂ and pH as indicated by Phenol Red is between 7.2 and 7.4. This may be scaled up in larger culture flasks appropriately, keeping the density of cells (about 1×10⁶ cells/mL). Supernatant can be collected at 72±2 hours of culture or sooner, as determined to be appropriate by those of skill in the art. Supernatant can be spun down, e.g., at 2,000 g, to pellet cells and particulates. Supernatants can then be collected and aliquoted, and then stored at −80° C. until the day of use. On the day of use an aliquot can be thawed at 4° C. and stored at 4° C. in between administration of the drops. In other examples, the composition is administered by use of an ointment, gel, polymers, or by injection.

The cell culture supernatant can be used as a pDC secretome in therapy as is (i.e., as obtained directly by removal from pDC cell culture), or optionally after one or more treatment steps. For example, the supernatant may be UV treated, filter sterilized, etc. One or more purification steps may be employed. In particular, the supernatant may be concentrated, for example, by dialysis or ultrafiltration. For example, the supernatant may be concentrated using membrane ultrafiltration with a nominal molecular weight limit (NMWL) of for example 3K. As shown in experiments set forth below, the secretome can function in the presence of anti-NGF antibodies, e.g., in the context of NCP.

The compositions of the invention can optionally be comprised within vessels that facilitate administration, e.g., eye dropper bottles, or bottles or tubes from which the compositions can be removed using an eye dropper. Optionally, the compositions can comprise a preservative and/or another therapeutic agent (e.g., as described herein). The compositions can be in liquid form or can be present in a gel or ointment. The compositions can further be included within a kit that includes the compositions (e.g., as described herein) and optionally one or more device for use in administration.

Compositions of the invention are advantageously administered to subjects by eye drop, with the amount and frequency of administration used being determined to be appropriate by those of skill in the art based on factors such as, for example, the condition to be treated, the severity of the condition, the age and general health of the subject, and the concentration of therapeutic agents in the medium. Treatment according to the methods of the invention thus can take place just once, or can be repeated (e.g., 2, 3, 4, 5, 6, 7, 8, 9, 10, or more times). In the case of multiple treatments, appropriate intervals between treatments can be selected by those of skill in the art. The invention thus includes, e.g., hourly, twice daily, thrice daily, daily, weekly, monthly, bi-monthly, semi-annual, or annual treatments.

In some examples, the compositions are administered dropwise (e.g., 1, 2, 3, 4, or 5 drops), 1-5 (e.g., 1, 2, 3, 4, or 5) times per day, every other day, or 1, 2, 3, 4, 5, 6, or 7 days per week. In one example, one drop is administered three time per day. Treatment can be carried out for 1, 2, 3, 4, 5, 6, or 7 days, or for 1, 2, 3, 4, 5, or 7 weeks, as determined to be appropriate by those skilled in the art and based on, e.g., the factors described above (e.g., the condition to be treated, etc.) as well as improvements obtained by the treatment. Accordingly, if a subject's condition is improved to a comfortable level or reaches a clinically relevant standard, treatment can be stopped.

In addition to administration by eye drops, other approaches known in the art for ophthalmic administration can be used, as determined to be appropriate by those of skill in the art. Different routes of administration are utilized, depending upon the part of the eye to be treated. For example, for treatment of a disease or condition of the cornea, direct topical application of a formulation (e.g., as described above) to the cornea can be used. For treatment of a disease or condition of another part of the eye, e.g., the retina or the choroid, a different approach to administration may be selected. For example, intravitreal or sub-retinal injection may be utilized as determined to be appropriate by those of skill in the art.

Treatment with pDC secretome compositions can be carried out to prevent, reduce, or eliminate one or more symptoms or features of a disease or condition of the eye (e.g., as described herein) by, for example, 10% or more (e.g., at least 10%, 20%, 30%, 40%, 50%, 60%, 70%, 80%, 90%, or 100%) as compared to the amount of one or more symptoms or features before treatment. For example, the symptom or feature can be reduced by 25%, 50%, 2-fold, 5-fold, 10-fold or more, or is eliminated. Improvements in symptoms or features of the disease or condition may be assessed clinically by those of skill in the art.

In some examples, administration of pDC secretome compositions treats a disorder or condition of the eye by reducing nerve degeneration or damage (e.g., corneal nerve damage). Nerve regeneration (e.g., recovery from nerve damage) can be enhanced by, for example, 10% or more (e.g., at least 10%, 20%, 30%, 40%, 50%, 60%, 70%, 80%, 90%, or 100%) as compared to the baseline nerve density prior to treatment. For example, nerve regeneration can be enhanced by 25%, 50%, 2-fold, 5-fold, 10-fold or more. In other examples, further nerve degeneration or damage in a subject is stopped.

In further examples, administration of pDC secretome compositions treats a disorder or condition of the eye by reducing inflammation within or around the eye. Inflammation can be reduced by, for example, 10% or more (e.g., at least 10%, 20%, 30%, 40%, 50%, 60%, 70%, 80%, 90%, or 100%) as compared to the baseline inflammation prior to treatment.

Symptoms and features of eye disease that can be improved according to the invention can be assessed, for example., visually, e.g., by in vivo confocal imaging, or by restoration of function, such as increased tear production and secretion, improved wound healing, reduced pain, improved vision, and improved reflexes, such as the corneal blink reflex.

In the case of prophylactic treatment, subjects at risk of developing a disease or condition of the eye, as described herein (e.g., subjects at risk for DED, NCP, COSP, NK, corneal neovascularization, persistent corneal epithelial defect, and other diseases and conditions characterized by ocular nerve degeneration or damage, intraocular inflammation due to a disease or condition of the eye, and/or corneal, retinal, or choroidal neovascularization), may be treated prior to symptom onset or when symptoms first appear, to prevent development or worsening of the DED, NCP, COSP, NK, corneal neovascularization, persistent corneal epithelial defect, nerve degeneration or damage, inflammation, or neovascularization. For example, in subjects already presenting with DED, NCP, COSP, NK, corneal neovascularization, or persistent corneal epithelial defect further development of symptoms can be prevented by the methods of the invention. Similarly, in subjects already presenting with nerve damage or degeneration, further damage or degeneration can be prevented by use of the methods and compositions of the invention. Also, in subjects already presenting with symptoms of intraocular inflammation, further inflammation can be prevented using the methods and compositions of the invention. Furthermore, in subjects already presenting with neovascularization, further growth of vessels into presently avascular tissue can be prevented by the methods of the present invention.

Optionally, the method of the invention can be carried out in combination with other methods for treating the disease or condition that the subject may have. The compositions of the invention can be administered concomitantly or sequentially with respect to the other methods, as determined to be appropriate by those of skill in the art.

In some embodiments, a subject treated according to the methods of the present invention is also treated with one or more TLR7 agonist and/or a TLR9 agonist. TLR7 agonists that can be used in the invention include, e.g., imiquimod, resiquimod, gardiquimod, loxoribine, vesatolimod (GS-9620), and GS-986. As is understood in the art, some TLR7 agonists (e.g., gardiquimod, imiquimod, and resiquimod) are also TLR8 agonists. Use of such dual agonists (e.g., TLR7/8 agonists) are also included within the scope of the invention. TLR9 agonists that can be used in the invention include CpG oligonucleotides. In specific non-limiting examples, the following agents can be used: CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10.

The agonists can be administered in amounts determined to be appropriate by those of skill in the art. Exemplary amounts of TLR7 (or TLR7/8) agonists for administration are one or more drops (e.g., 1, 2, 3, 4, or 5 drops) of a 0.05-10% w/v (e.g., 0.1-8%, 1-6%, 2-5%, or 3-4% w/v) solution, while exemplary amounts of TLR9 agonists are 0.5-100 mg (e.g., 1-75, 3-50, 5-40, 10-30, or 15-25 mg) per dose. Optionally, the TLR7 (including TLR7/8 agonists) and TLR9 agonists are comprised within pharmaceutically acceptable compositions, such as ophthalmic compositions, as known in the art. The agonists are included within these compositions in amounts sufficient to provide a desired dosage, using a desired volume (e.g., the volume of a drop from a standard eye dropper), as can be determined by those of skill in the art.

In other examples, the pDC secretome compositions are administered with one or more anti-angiogenic agents and/or nerve growth factor (NGF).

In addition to including methods of combination therapy, such as those described above, the invention also includes combination compositions as well as kits that include one or more agent as described herein.

The invention also provides method for generating the compositions described herein. In these methods, cells are cultured in cell culture medium, e.g., as described herein, and supernatants from the cells are obtained. Exemplary conditions and materials used in the production methods are described above.

### Identification of Subjects

As noted above, the compositions and methods of the invention can advantageously be used in the treatment of dry eye disease (DED), neuropathic corneal pain (NCP), chronic ocular service pain (COSP), neurotrophic keratopathy (NK), corneal neovascularization, and persistent corneal epithelial defect. More generally, subjects who can be treated using the methods and compositions of the invention include those suffering from, or at risk for, nerve degeneration or damage, inflammation of the eye, and/or neovascularization. The subjects include human patients (adults and children) who have or are at risk of developing a disease or condition of the eye as described herein. In other embodiments, the subject is a veterinary patient, e.g., a dog or a cat, which may be experiencing a disease or condition described herein (e.g., dry eye disease). In these embodiments, the pDCs may be obtained from a corresponding veterinary source (e.g., blood isolated from an allogenic or autologous sample).

The cornea is the most densely innervated structure in the human body, and is therefore highly sensitive to touch, temperature, and chemical stimulation, all of which are sensed by corneal nerves. Corneal nerves are also involved in blinking, wound healing, and tear production and secretion. Damage to or loss of corneal nerves can lead to dry eyes, impairment of sensation, corneal edema, impairment of corneal epithelium healing, corneal ulcerations and erosions, and a cloudy corneal epithelium, among other conditions. Diseases or conditions characterized by corneal nerve degeneration or damage include, for example, dry eye disease (DED), neurotrophic keratitis, corneal infections, excessive or improper contact lens wear, ocular herpes (HSV), herpes zoster (shingles), chemical and physical burns, injury, trauma, surgery (including corneal transplantation, laser assisted in-situ keratomileusis (LASIK), penetrating keratoplasty (PK), automated lamellar keratoplasty (ALK), photorefractive keratectomy (PRK), radial keratotomy (RK), cataract surgery, and corneal incisions), abuse of topical anesthetics, topical drug toxicity, corneal dystrophies, vitamin A deficiency, diabetes, microbial keratitis, and herpetic keratitis (caused by, e.g., HSV-1). The methods and compositions of the invention can be used to prevent or treat any of the aforementioned diseases or conditions of the eye.

Patients having or at risk of developing diseases or conditions characterized by inflammation within the eye can also be treated using the methods and compositions of the invention. Thus, for example, patients having or at risk of the following diseases or conditions can be treated: episcleritis, scleritis, uveitis (e.g., anterior uveitis (including iritis and iridocyclitis), intermediate uveitis (including vitritis and pars planitis), posterior uveitis (including retinitis, choroiditis, chorioretinitis, and neuroretinitis), panuveitis (infectious) (including endophthalmitis), and panuveitis (non-infectious)), and retinal vasculitis.

Neovascularization is a common feature of many conditions, and may occur in tissues of the eye including, for example, the cornea, retina, or choroid. This process involves new blood vessel formation in abnormal locations, such as the cornea, a normally avascular tissue. Diseases that are characterized by corneal neovascularization include, for example, corneal infection, inflammation, autoimmune disease, limbal stem cell deficiency, neoplasia, dry eye disease, radiation, blepharitis, uveitis, keratitis, corneal ulcers, glaucoma, rosacea, and lupus. Trauma, such as surgery, injury, burn (e.g., chemical burn), injury, and excessive or improper contact lens use, can also be characterized by neovascularization. Inflammation associated with ocular (e.g., corneal) neovascularization can result from bacterial and viral infection, Stevens-Johnson syndrome, graft rejection, ocular cicatricial pemphigoid, and degenerative disorders, such as pterygium and Terrien marginal degeneration. Diseases or conditions that are characterized by retinal neovascularization include, for example, ischemic retinopathies, diabetic retinopathy, retinopathy of prematurity, retinal vein occlusions, ocular ischemic syndrome, sickle cell disease, radiation, and Eales' disease. Further, diseases or conditions that are characterized by choroidal neovascularization include, for example, inflammatory neovascularization with uveitis, macular degeneration, ocular trauma, trauma due to excessive or improper contact lens wear, sickle cell disease, pseudoxanthoma elasticum, angioid streaks, optic disc drusen, extreme myopia, malignant myopic degeneration, and histoplasmosis. Subjects having or at risk of developing any of the aforementioned disorders or conditions can be treated using the methods and compositions of the invention.

TABLE 1
RPMI 1640 Medium

| Inorganic salts | Amino acids | Vitamins | Other |
|---|---|---|---|
| 100 mg/L of Ca(NO₃)₂•4H₂O | 200.00 mg/L L-Arginine | 0.20 mg/L Biotin | 2000.00 mg/L |
| 400 mg/L KCl | 56.82 mg/L L-Asparagine•H₂O | 0.25 mg/L D- | D-Glucose |
| 48.80 mg/L | 20.00 mg/L L-Aspartic acid | Calcium | 1.00 mg/L Glutathione |
| MgSO₄ (anhydrous) | 65.20 mg/L L-Cystine•2HCl | pantothentate | (reduced) |
| 6000.00 mg/L NaCl | 20.00 mg/L L-Glutamic Acid | 3.00 mg/L Choline | 5.00 mg/L Phenol |
| 800.70 mg/L | 300.00 mg/L L-Glutamine | chloride | red•Na |
| Na₂HPO₄ (anhydrous) | 10.00 mg/L Glycine | 1.00 mg/L Folic | |
| 2000.00 mg/L NaHCO₃ | 15.00 mg/L L-Histidine | Acid | |
| | 20.00 mg/L Hydroxy-L-proline | 35.00 mg/L/— | |
| | 50.00 mg/L L-Isoleucine | Inositol | |
| | 50.00 mg/L L-Leucine | 1.00 mg/L | |
| | 40.00 mg/L L-Lysine•HCl | Nicotinamide | |
| | 15.00 mg/L L-Methionine | 1.00 mg/L Para- | |
| | 15.00 mg/L L-Phenylalanine | Aminobenzoic acid | |
| | 20.00 mg/L L-Proline | 1.00 mg/L | |
| | 30.00 mg/L L-Serine | Pyridoxine•HCl | |
| | 20.00 mg/L L-Threonine | 0.20 mg/L | |
| | 5.00 mg/L L-Trptyophan | Riboflavin | |
| | 28.83 mg/L L-Tyrosine•2Na•2H₂O | 1.00 mg/L | |
| | 20.00 mg/L L-Valine | Thiamine•HCl | |
| | | 0.005 mg/L Vitamin | |
| | | B₁₂ | |

### EXAMPLES

### Example 1

The experiments described in this example show that the pDC secretome promotes corneal nerve survival and that pDCs modulate cold receptor function.

The cornea has a rich innervation and is also known to contain resident immune cells. Neuro-immune crosstalk has gained interest for its role in health and disease. Plasmacytoid dendritic cells (pDCs) are present within the cornea and associate closely with corneal nerves. We have demonstrated that selective depletion of corneal pDCs leads to a rapid loss of corneal nerves. We have found that pDCs are key mediators of neuro-immune crosstalk within the cornea. Using C57BL/6-Tg (CLEC4C-HBEGF)956CIn/J mice (Swiecki et al., Immunity 33(6):955-966, 2010) (hereafter, pDC-DTR; available from Jackson Labs, bred to homozygous), which express DTR, and by the selective depletion of corneal pDCs, we demonstrate that: (i) the pDC secretome possesses neurotrophic properties, as treatment with pDC supernatant preserves corneal nerves, and (ii) corneal cold receptors become dysfunctional in the absence of pDCs. In this example, we show the neurotrophic properties of the pDC secretome. Furthermore, we characterize functional alterations of corneal nerves by ex vivo electrophysiology.

### Methods

### pDC Sorting and Supernatant Collection

Splenic pDCs were sorted from 6-8 weeks old C57BL/6J mice using a BD Biosciences Aria II Cell Sorter. After excluding debris, doublets, and dead cells, pDCs were identified as the CD45+B220+PDCA-1+SiglecH+ and F4/80-population. pDCs were cultured in serum-free RPMI 1640 medium for 3 days, at which point the supernatant was collected consisting of the pDC secretome.

### Rescue Studies With pDC Secretome

6-8 weeks old C57BL/6J (Jackson Labs) and pDC-DTR mice (Swiecki, supra) were used for these experiments. Groups were as indicated below in Table 2, such that mice received subconjunctival injections of diphtheria toxin (30 ng/eye) or normal saline (for the sham depleted group) at Day 0 and repeated every 2 days, and serum-free media or pDC supernatant was applied topically to the cornea for one week, beginning at Day 1. At Day 7, corneas were excised and underwent immunofluorescent staining for BIII-tubulin to assess corneal innervation by confocal microscopy. Corneal nerve density was analyzed in ImageJ using the NeuronJ plugin.

TABLE 2
Groups and Treatments for Rescue Studies

| | | Diphtheria | Serum-Free | pDC |
|---|---|---|---|---|
| Group | Strain | Toxin | Media | Supernatant |
| WT + DT | C57BL/6J | + | − | − |
| Sham Depl. | pDC-DTR | − | − | − |
| pDC Depl. | pDC-DTR | + | − | − |
| Depl. + Media | pDC-DTR | + | + | − |
| Depl. + Sup. | pDC-DTR | + | − | + |

### Ex Vivo Corneal Electrophysiology

6-8 weeks old C57BL/6J (Jackson Labs) and pDC-DTR mice (Swiecki, supra) were used for these experiments. These experiments included the WT+DT, Sham Depl., and pDC Depl. groups. Eyes were collected in physiologic solution (pH 7.2-7.4) and allowed to adapt for 30 minutes prior to starting recordings. Recordings were conducted using an Ag/AgCl recording electrode with a finely pulled pipette tip (˜20 μm diameter). (Carr et al., J. Gen. Physiol. 121:427-439, 2003; Madrid et al., J. Neurosci. 29:3120-3131, 2009). Baseline firing rate was recorded for 2 minutes before beginning cooling, with a return to physiologic temperature for at least 2 minutes between temperature ramps (Carr, supra; Madrid, supra). The raw data was filtered and amplified using the respective modules (Digitimer). Data was acquired using the Spike2 software package (v9.4). The following parameters were assessed: basal firing rate, cooling threshold, cooling response, and a post: pre ratio of the firing rate before and after the cooling response as a measure of possible nerve terminal exhaustion.

### Results

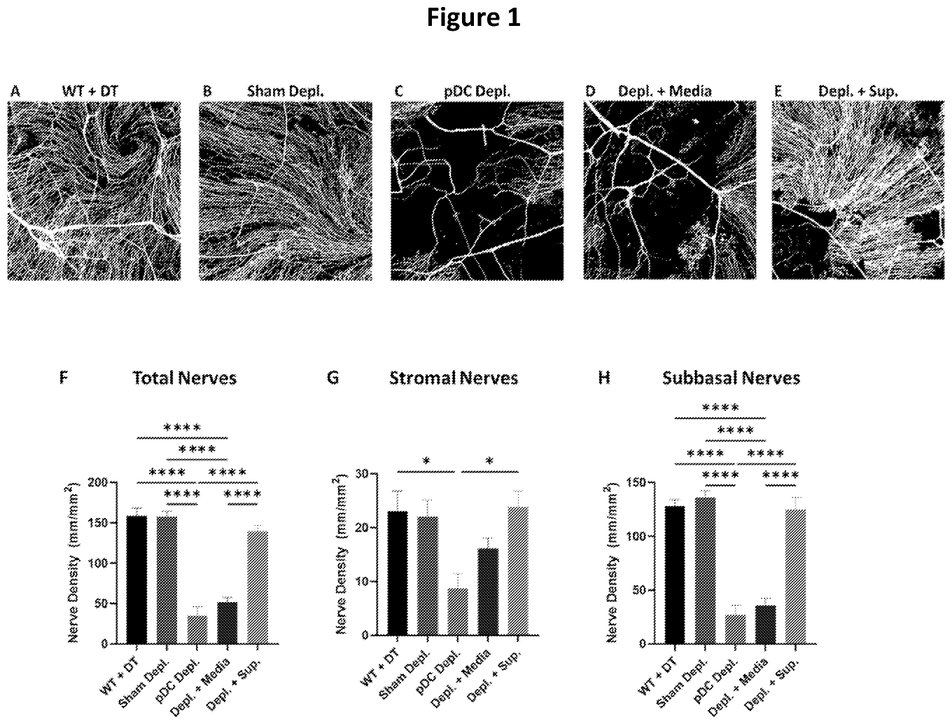

FIG. 1 shows that topical application of the pDC secretome rescues the corneal nerve loss following pDC depletion. Representative confocal micrographs of corneal whole mounts stained with Bill-tubulin, a nerve-specific marker, are shown in FIG. 1 (A-E). Images were taken in the central cornea with a Nikon A1r confocal microscope. Note the dense innervation in the WT+DT (A) and Sham Depl. (B) groups, whereas this is lost following pDC depletion (C). Application of serum-free media fails to restore the innervation (D), whereas application of the pDC secretome partially rescues the innervation (E). Quantification of the total (F), stromal (G), and subbasal nerves (H) is shown. Statistical analysis was performed by One-Way ANOVA followed by Tukey's multiple comparisons test for ANOVA p-values <0.05; *, p<0.05; **, p<0.01; ***, p<0.001; ****, p<0.0001. Scale bar is 100 μm. Nerve density values from rescue studies are provided in Table 3.

TABLE 3
Nerve Density Values from Rescue Studies

| | Total Nerves | Stromal Nerves | Subbasal nerves |
|---|---|---|---|
| Group | (mm/mm2) | (mm/mm2) | (mm/mm2) |
| WT + DT | 158.60 ± 10.02 | 23.10 ± 3.72 | 128.00 ± 6.06 |
| Sham Depl. | 158.10 ± 6.76 | 22.15 ± 3.04 | 136.00 ± 6.54 |
| pDC Depl. | 35.59 ± 10.76 | 8.80 ± 2.65 | 26.79 ± 9.68 |
| Depl. + Media | 52.06 ± 5.93 | 16.17 ± 1.94 | 35.90 ± 6.80 |
| Depl. + Sup. | 140.2 ± 6.95 | 23.85 ± 2.99 | 125.2 ± 11.13 |

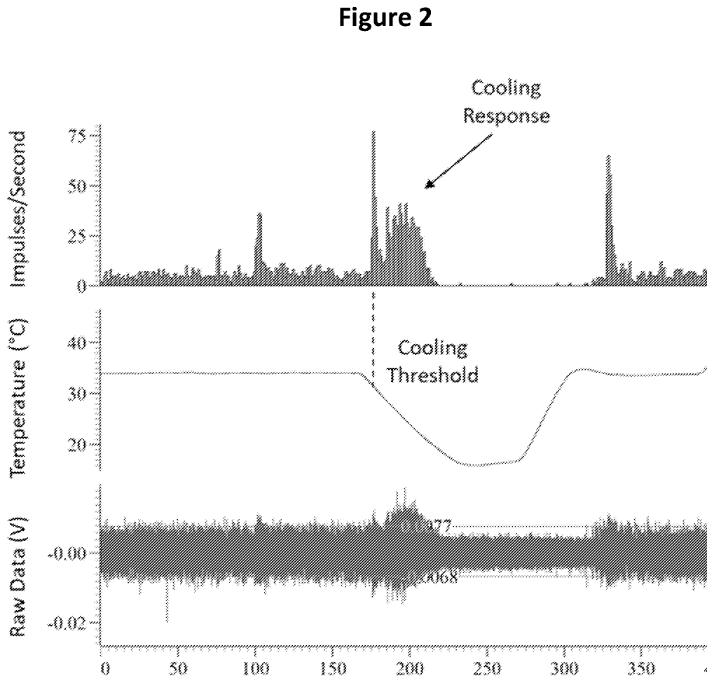

FIG. 2 shows sample trace of ex vivo corneal electrophysiology. The bottom trace in FIG. 2 represents the raw, unfiltered data. The middle trace indicates the temperature throughout the recording interval. The top trace is representative of the filtered data represented in terms of impulses/second. The cooling response and cooling threshold are also indicated.

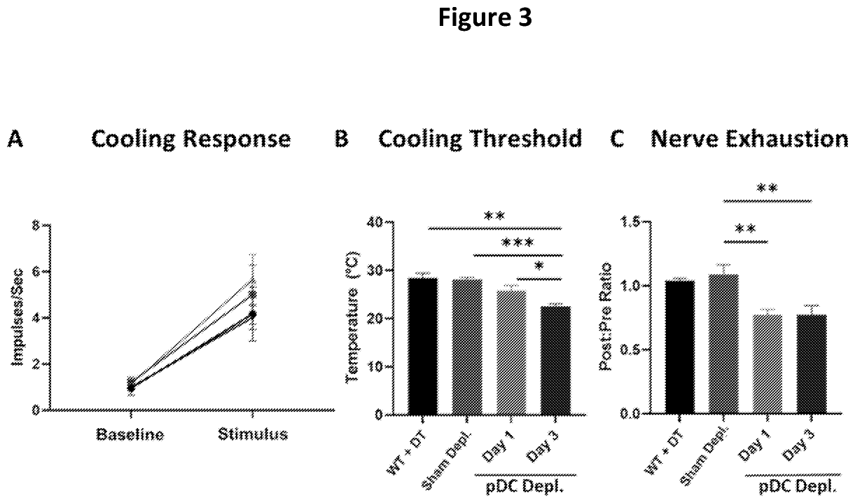

FIG. 3 shows electrophysiological recordings of corneal nerve terminal impulses, which reveal dysfunction of high-threshold cold receptors following pDC depletion. (A) Cooling responses from WT+DT, Sham Depletion, and 1 and 3 days of pDC depletion reveal no difference in baseline firing or response to stimulus. (B) The cooling threshold, the temperature at which nerve terminals respond to the cooling stimulus, is decreased following pDC depletion. (C) The post: pre ratio of firing with respect to the cooling stimulus is also decreased, possibly indicating nerve exhaustion. Statistical analysis was performed by One-Way ANOVA followed by Tukey's multiple comparisons test for ANOVA <0.05; *, p<0.05; **, p<0.01; ***, p<0.001.

### Conclusion

This study is the first, to our knowledge, to demonstrate neurotrophic properties of the pDC secretome. Structural alterations of corneal nerves following pDC depletion are significantly reversed by application of pDC supernatant. Our study also uncovered dysfunction of high-threshold cold receptors in the cornea after pDC depletion, further supporting the key role of pDCs in corneal neuro-immune crosstalk. These findings may have implications for ocular diseases in which nerve abnormalities predominate.

### Example 2

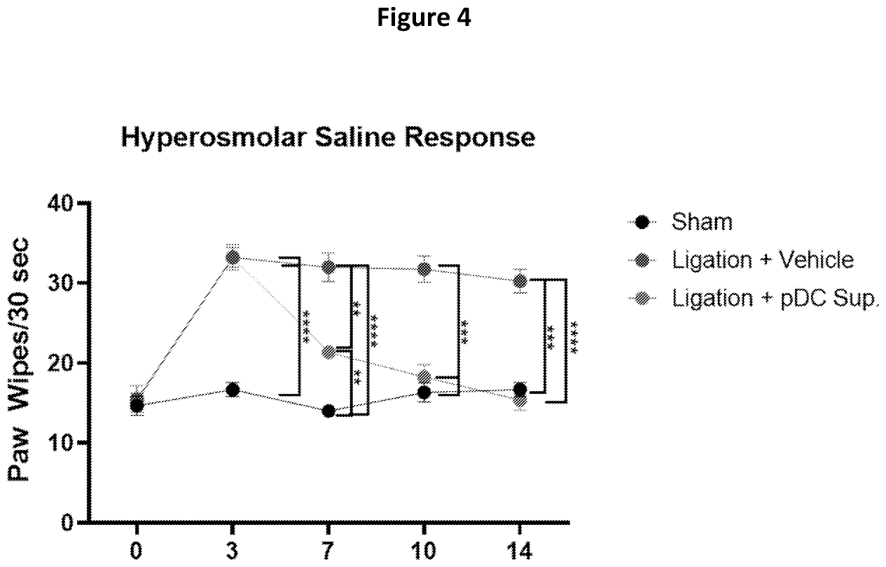

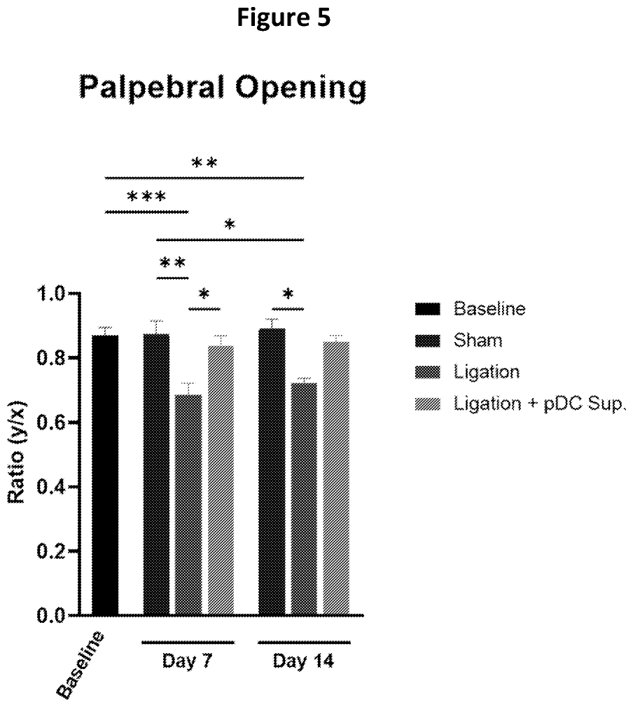

In additional studies, we examined the effects of pDC supernatant on hyperosmolar saline response (FIG. 4) and palpebral opening (FIG. 5) in a ciliary nerve ligation induced neuropathic corneal pain (NCP) model.

FIG. 4 shows hypersensitivity responses to 5M saline following ciliary nerve ligation or sham surgery. Sham animals had no significant increase in response to 5M saline, whereas ligated animals had a significant increase in response at Day 3. The initiation of treatment began at Day 3, and mice received either Vehicle Treatment or pDC Supernatant. Vehicle treatment had no effect on the hypersensitivity response, whereas treatment with pDC Supernatant did reduce this response as early as Day 7, with a return to baseline by Day 14.

FIG. 5 shows palpebral opening following ciliary nerve ligation as a measure of spontaneous pain. Palpebral opening can be used to assess spontaneous pain in mice following injury to the ocular surface. This depends on the ratio of the distance between the upper and lower lids (y) and the distance between nasal and temporal canthi (x). The ratio gives an indication of the exposure of the ocular surface to the external environment, so an animal in more pain would have a decreased ratio indicating that less of the eye was exposed. Sham surgery has no effect on this ratio, whereas following ligation there is a decrease in the ratio compared to baseline. Treatment with pDC supernatant improves this ratio, indicative of less spontaneous pain.

### Example 3

Mice exposed to dessicating stress developed clinical signs of dry eye disease (DED). For example, corneal nerve density decreased in the murine DED model at day 14. mRNA expression analysis revealed that expression of neurotrophins was downregulated. This was confirmed by ELISA protein expression analysis, which showed that expression of neurotrophins decreased significantly in DED at day 14. Furthermore, mRNA expression of neuropeptides was downregulated in corneas and a significant up-regulation in substance P (SP) and calcitonin gene-related peptide (CGRP) may reflect the alteration of innervation in DED. Changes in mRNA expression were also found in TG following corneal nerve damage implicated in the development of neurogenic inflammation.

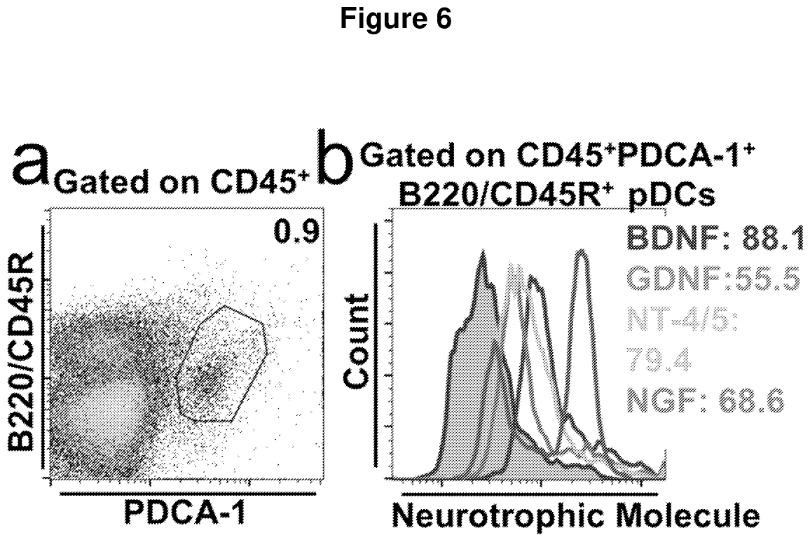

Plasmacytoid dendritic cells (pDCs) express neurotrohic molecules (FIG. 6). We investigated whether neuromediators released by pDCs could provide trophic support to the ocular surface with the potential to reverse DED. To test this, 2×10⁶ splenic GFP+pDCs were sorted and cultured for 3 days (serum free). Then, supernatant was harvested and applied onto the ocular surface 4×/day for 7 days (immediately after induction of DED, 7 days). Medium only was used as a control.

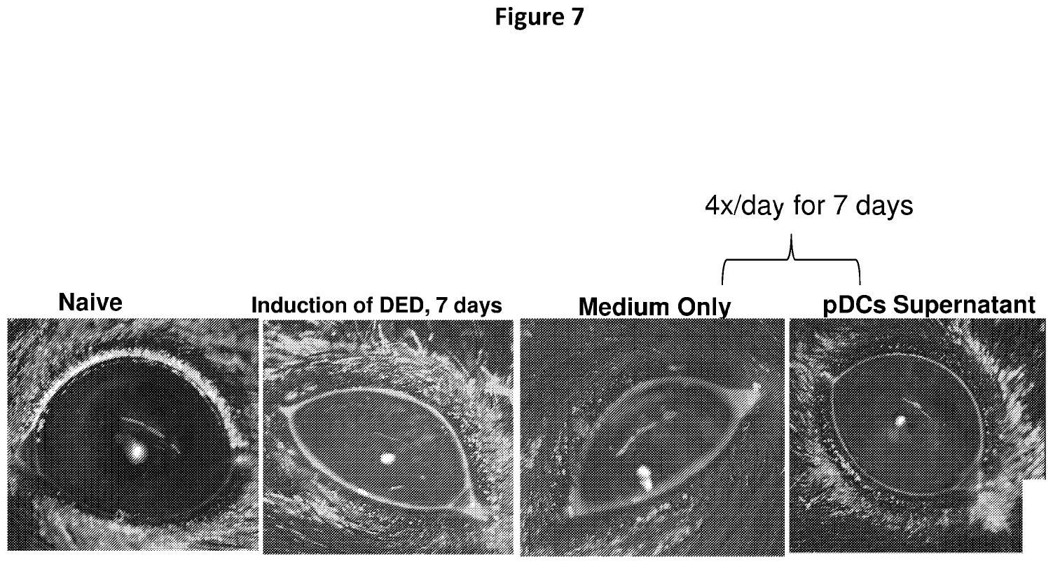

Clinical evaluations included measurement of tear secretion volume (cotton thread test), corneal fluorescein staining, corneal mechanical sensitivity (blink test) with Cochet-Bonnet, and eye wipes (corneal sensory) based on treatment with10 uL of hyperosmolar 5M saline. Tissue was harvested and neurotrophins were assessed by qRT-PCR for cornea. The results are set forth in FIG. 7, which shows (i) a naïve, untreated eye, (ii) an eye after induction of DED, 7 days, (iii) an eye treated with medium only 4×/day for 7 days, and (iv) an eye treated with pDC supernatant 4×/day for 7 days. In contrast to the medium only treatment, which showed a phenotype similar that to the DED eye, the pDC supernatant-treated eye showed improvement getting close to that of the naïve eye.

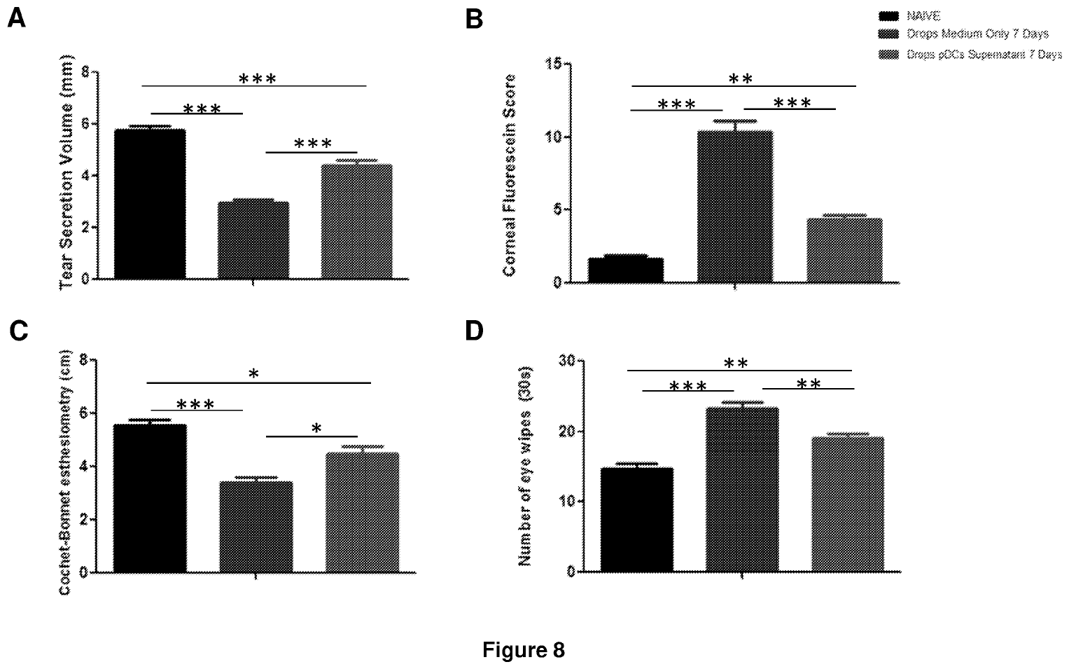

Various features of the ocular surfaces of desiccating stress-induced dry eye disease in mice treated with medium vs pDC supernatant eye drops were analyzed. These features include tear secretion volume, corneal fluorescein score, Cochet-Bonnet estheslometry, and number of eye wipes. FIG. 8A shows the results of tear volume analysis using a phenol red thread test. The results show a significant decrease in tear volume in mice exposed to desiccating stress and treated with medium only eye drops as compared to naïve controls. Treatment with pDC supernatant drops improved the tear secretion volume. FIG. 8B shows quantification of fluorescein staining scores, which revealed a significant increase in corneal staining in mice exposed to desiccating stress in mice treated with medium only eye drops as compared to naïve controls. Treatment with pDC supernatant drops improved the corneal fluorescein score. FIG. 8C shows that corneal mechanical sensitivity decreased in mice treated with medium only, and FIG. 8D shows that nerve dysfunctions markedly exacerbated the response in mice treated with medium only eye drops as compared to the control. Improvements in both of these features was obtained by treatment with pDC supernatant. Results are presented as average±SEM.

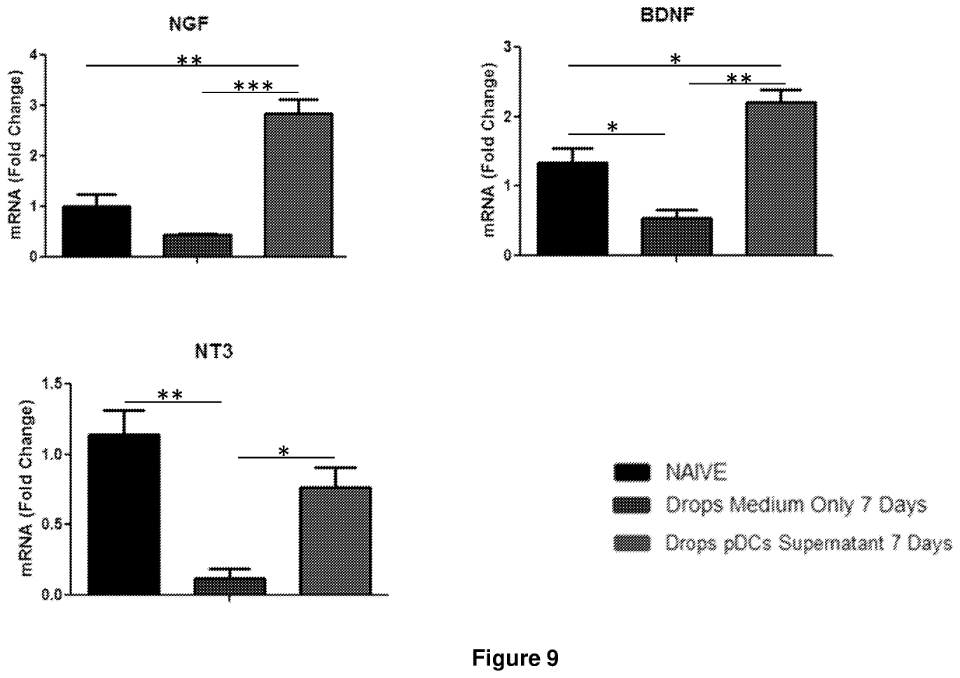

Neurotrophin mRNA in corneas was measured by qRT-PCR analysis. As shown in FIG. 9, NGF, BDNF, and NT3 were downregulated in corneas of mice treated with medium-only eye drops. In contrast, NGF and BDNF were upregulated in corneas of mice treated with pDC supernatant eye drops. The results are presented at fold changes normalized to the naïve controls group. Fold changes are presented as average±SEM.

Our results showed that topical administration of pDC supernatant (serum free) had beneficial effects. For example, the results show that pDC supernatant can be used as a topical therapeutic strategy to reverse dry eye disease. These beneficial effects can be obtained without debridement the central epithelium, which may be required for the use of other approaches, such as cell-based therapies. Clinical evaluations demonstrated that mice exposed to desiccating stress the clinical signs of DED showed improvement as compared to the medium only treatment. Furthermore, there was downregulation by mRNA expression for neurotrophins in corneas with medium only eye drops and a significant up-regulation in NGF and BDNF by treatment with pDC supernatant.

### Example 4

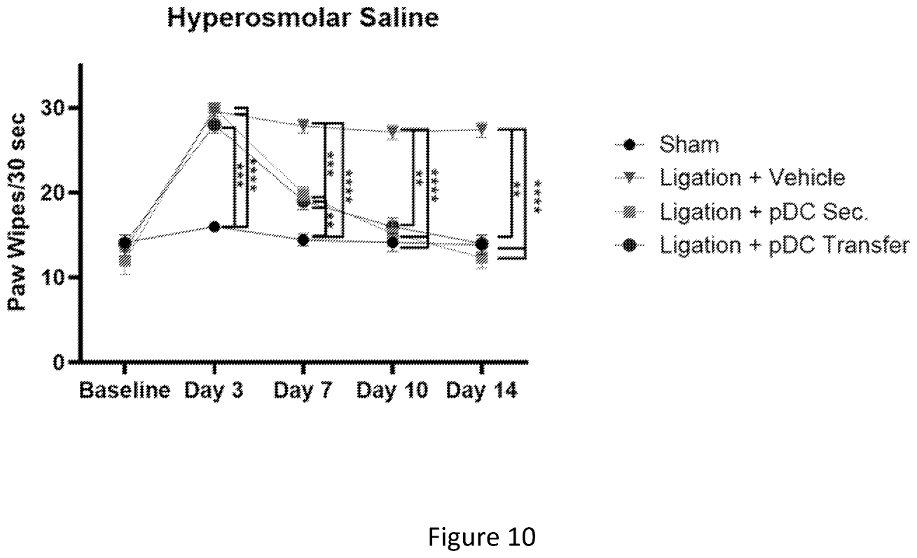

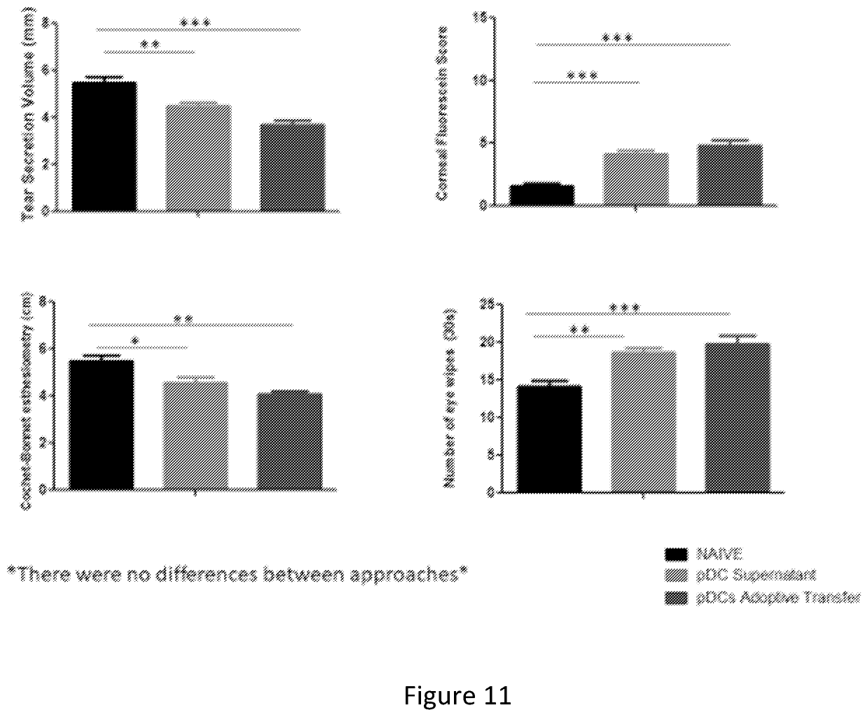

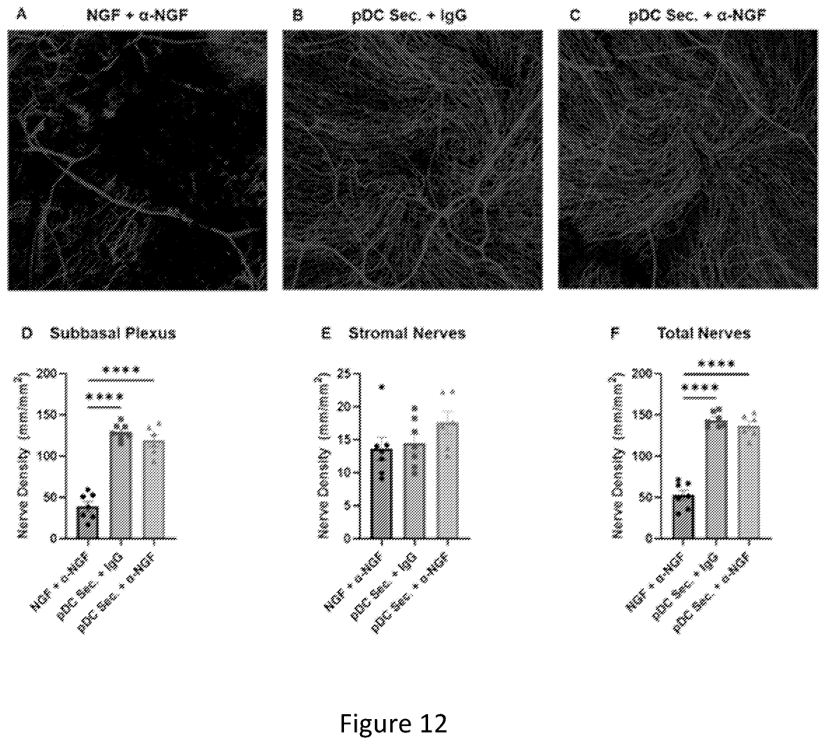

Hypersensitivity responses to 5M saline following sham surgery and ciliary nerve ligation with or without treatments were assessed, and the results are shown in FIG. 10. Sham animals had no significant increase in response to 5M saline, whereas ligated animals had a significant increase in response at Day 3. The initiation of treatment began at Day 4, and mice received either vehicle treatment, pDC secretome, or pDCs by local adoptive transfer to the cornea. Vehicle treatment had no effect on the hypersensitivity response, whereas treatment with pDC secretome or pDC transfer led to a reduction in the response by Day 7 with a return to normal values by Day 10 (when sham and treated ligation animals no longer differ) and persisting until Day 14. Two-Way ANOVA with repeated measures, followed by Tukey's multiple comparisons test; *, p<0.05; **, p<0.01; ***, p<0.001; ****, p<0.0001. In additional studies, shown in FIG. 11, equivalency of pDC secretome vs. pDC adoptive transfer was achieved in a DED model. In further experiments, it was shown by use of anti-NGF antibodies administered with a pDC secretome composition that NGF is not a required component of the secretome in NCP (FIG. 12).

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein.

All publications and patent applications mentioned in this specification are herein incorporated by reference to the same extent as if each independent publication or patent application was specifically and individually indicated as being incorporated by reference in their entirety.

Use of singular forms herein, such as “a” and “the,” does not exclude indication of the corresponding plural form, unless the context indicates to the contrary. Similarly, use of plural terms does not exclude indication of a corresponding singular form.

Some embodiments are within the scope of the following numbered paragraphs.
1. A method of treating a disease or condition of the eye in a subject, the method comprising administering a plasmacytoid dendritic cell (pDC) secretome to the eye of the subject.
2. A method of increasing nerve density in the eye of a subject, the method comprising administering a pDC secretome to the eye of the subject.
3. A method of improving corneal nerve function in a subject, the method comprising administering a pDC secretome to the eye of the subject.
4. A method of promoting corneal nerve survival in a subject, the method comprising administering a pDC secretome to the eye of the subject.
5. A method of treating a subject who has undergone corneal transplantation, the method comprising administering a pDC secretome to the eye of the subject post transplantation to prevent rejection.
6. The method of any one of paragraphs 1 to 5, wherein the method prevents, reduces, or eliminates one or more symptoms of a disease or condition of the eye in the subject.
7. The method of any one of paragraphs 1 to 6, wherein the subject has or is at risk of developing dry eye disease (DED), neuropathic corneal pain (NCP), chronic ocular service pain (COSP), neurotrophic keratopathy (NK), corneal neovascularization, persistent corneal epithelial defect, or one or more other diseases or conditions characterized by ocular nerve degeneration or damage, ocular inflammation, or ocular neovascularization (e.g., as described herein).
8. The method of any one of paragraphs 1 to 7, wherein the pDC secretome composition is administered to the eye of the subject by eye drops, ointment, gel, polymers, or injection.
9. The method of any one of paragraphs 1 to 8, wherein the method does not comprise debridement of the corneal surface of the eye.
10. The method of any one of paragraphs 1 to 9, wherein the pDC secretome is acellular.
11. The method of any one of paragraphs 1 to 10, wherein the pDC secretome comprises one or more of brain-derived neurotrophic factor (BDNF), glial cell derived neurotrophic factor (GDNF), neurotrophin-4/5 (NT-4/5), nerve growth factor (NGF), anti-inflammatory molecules, and anti-angiogenic molecules.
12. The method of paragraph 11, wherein the pDC secretome comprises each of BDNF, GDNF, NT-4/5, NGF, anti-inflammatory molecules, and anti-angiogenic molecules.
13. The method of any one of paragraphs 1 to 12, further comprising administration of an additional therapeutic agent to the eye of the subject.
14. The method of paragraph 13, wherein the additional therapeutic agent is a TLR7 agonist, a TLR9 agonis, or nerve growth factor (NGF).
15. The method of any one of paragraphs 1 to 14, wherein the subject is a human.
16. The method of any one of paragraphs 1 to 14, wherein the subject is a veterinary subject, such as a dog or a cat.
17. The method of any one of paragraphs 1 to 16, wherein the pDC secretome comprises molecules secreted from pDCs cultured in a cell culture medium, and said cell culture medium (cell culture supernatant) comprising said molecules is administered to said subject.
18. A pharmaceutical composition comprising a pDC secretome, which optionally is in dosage form for administration to a subject.
19. The pharmaceutical composition of paragraph 18, wherein the pDC secretome comprises molecules secreted from pDCs cultured in a cell culture medium, and said pDC secretome is present within said cell culture medium (cell culture supernatant).
20. A pharmaceutical kit comprising a pDC secretome in dosage form, and optionally a vessel and/or device for use in administration (e.g., an eye dropper).
21. The pharmaceutical kit of paragraph 20, wherein the pDC secretome comprises molecules secreted from pDCs cultured in a cell culture medium, and said pDC secretome is present within said cell culture medium (cell culture supernatant).
22. A method of generating a pDC secretome for use in treating a disease or condition of the eye, the method comprising culturing pDCs in a medium and removing the pDCs from the medium.
23. The method of paragraph 22, further comprising collecting said medium for use as a composition comprising said pDC secretome in said treatment.
24. The method of paragraph 22 or 23, further comprising freezing or lyophilizing the medium after pDC removal.
25. Use of a pDC secretome composition, e.g., as described herein, for treating a disease or condition of the eye, e.g., as described herein.

Other embodiments are within the scope of the following claims.

What is claimed is:

1. A method of treating a disease or condition of the eye in a subject, the method comprising administering a plasmacytoid dendritic cell (pDC) secretome to the eye of the subject.

2. A method of increasing nerve density in the eye of a subject, the method comprising administering a pDC secretome to the eye of the subject.

3. A method of improving corneal nerve function in a subject, the method comprising administering a pDC secretome to the eye of the subject.

4. A method of promoting corneal nerve survival in a subject, the method comprising administering a pDC secretome to the eye of the subject.

5. A method of treating a subject who has undergone corneal transplantation, the method comprising administering a pDC secretome to the eye of the subject post transplantation to prevent rejection.

6. The method of claim 1, wherein the method prevents, reduces, or eliminates one or more symptoms of a disease or condition of the eye in the subject.

7. The method of claim 1, wherein the subject has or is at risk of developing dry eye disease (DED), neuropathic corneal pain (NCP), chronic ocular service pain (COSP), neurotrophic keratopathy (NK), corneal neovascularization, persistent corneal epithelial defect, or one or more other diseases or conditions characterized by ocular nerve degeneration or damage, ocular inflammation, or ocular neovascularization (e.g., as described herein).

8. The method of claim 1, wherein the pDC secretome composition is administered to the eye of the subject by eye drops, ointment, gel, polymers, or injection.

9. The method of claim 1, wherein the method does not comprise debridement of the corneal surface of the eye.

10. The method of claim 1, wherein the pDC secretome is acellular.

11. The method of claim 1, wherein the pDC secretome comprises one or more of brain-derived neurotrophic factor (BDNF), glial cell derived neurotrophic factor (GDNF), neurotrophin-4/5 (NT-4/5), nerve growth factor (NGF), anti-inflammatory molecules, and anti-angiogenic molecules.

12. The method of claim 11, wherein the pDC secretome comprises each of BDNF, GDNF, NT-4/5, NGF, anti-inflammatory molecules, and anti-angiogenic molecules.

13. The method of claim 1, further comprising administration of an additional therapeutic agent to the eye of the subject.

14. The method of claim 13, wherein the additional therapeutic agent is a TLR7 agonist, a TLR9 agonis, or nerve growth factor (NGF).

15. The method of claim 1, wherein the subject is a human.

16. The method of claim 1, wherein the subject is a veterinary subject, such as a dog or a cat.

17. The method of claim 1, wherein the pDC secretome comprises molecules secreted from pDCs cultured in a cell culture medium, and said cell culture medium (cell culture supernatant) comprising said molecules is administered to said subject.

18. A pharmaceutical composition comprising a pDC secretome, which optionally is in dosage form for administration to a subject.

19. The pharmaceutical composition of claim 18, wherein the pDC secretome comprises molecules secreted from pDCs cultured in a cell culture medium, and said pDC secretome is present within said cell culture medium (cell culture supernatant).

20. A pharmaceutical kit comprising a pDC secretome in dosage form, and optionally a vessel and/or device for use in administration (e.g., an eye dropper).

21. The pharmaceutical kit of claim 20, wherein the pDC secretome comprises molecules secreted from pDCs cultured in a cell culture medium, and said pDC secretome is present within said cell culture medium (cell culture supernatant).

22. A method of generating a pDC secretome for use in treating a disease or condition of the eye, the method comprising culturing pDCs in a medium and removing the pDCs from the medium.

23. The method of claim 22, further comprising collecting said medium for use as a composition comprising said pDC secretome in said treatment.

24. The method of claim 23, further comprising freezing or lyophilizing the medium after pDC removal.

25. Use of a pDC secretome composition, e.g., as described herein, for treating a disease or condition of the eye, e.g., as described herein.
